(19) United States
(12) Patent Application Publication
(10) Pub. No.: US 2026/0069191 A1
(43) Pub. Date: Mar. 12, 2026

(54) METHODS AND SYSTEMS FOR UNSUPERVISED ANALYSIS OF QRS COMPLEX CLASSIFICATIONS IN ECG SIGNALS

(71) Applicant: KONINKLIJKE PHILIPS N.V., Eindhoven (NL)

(72) Inventors: Joshua Michael Burns, San Diego (NL); Justin Tsoneshane Lee, San Diego, CA (US)

(21) Appl. No.: 19/319,827

(22) Filed: Sep. 5, 2025

Related U.S. Application Data
(60) Provisional application No. 63/691,344, filed on Sep. 6, 2024.

(51) Int. Cl. A61B 5/366

(52) U.S. Cl. CPC A61B 5/366

(57) ABSTRACT

A method for automated analysis of ECG signal classifications, comprising: receiving an ECG signal classification for a subject, comprising an ECG signal; analyzing the ECG signal classification to generate a final ECG signal classification, comprising: (i) filtering the received ECG signal; (ii) extracting features from each of a plurality of identified QRS complexes; (iii) clustering, using the extracted features, the identified QRS complexes into at least a first cluster of QRS complexes and abnormal or dissimilar QRS complexes; (iv) generating a QRS cluster template from the first cluster; (v) calculating a distance between each of the QRS complexes in the cluster of abnormal or dissimilar QRS complexes and the QRS cluster template; and (vi) classifying, based on the calculated distance, the QRS complexes in the cluster of abnormal or dissimilar QRS complexes as being normal or abnormal/dissimilar to generate the final ECG signal classification; and reporting the generated final ECG signal classification.

### FIELD OF THE DISCLOSURE

The present disclosure is directed generally to methods and systems for automated analysis of ECG signal classifications.

### BACKGROUND

The electrocardiogram (ECG) is a non-invasive technique that measures the electrical activity of the heart and is used to detect, among other things, cardiac disorders. Most commonly, ECG measurements are collected in a hospital or in an ambulatory setting. Ambulatory settings typically utilize either a Holter® monitor or mobile cardiac telemetry (MCT).

In addition to some clinician review, ECG signals are typically analyzed using one or more algorithms. It is not feasible to have medical personal to review all ECG records as the length of an ECG recording can be multiple days or even weeks long. Thus, ECG QRS complex and arrhythmia classification algorithms are typically used to screen these long ECG recordings and present findings to a clinician for review. ECG rule-based and deep learning algorithms often classify and annotate QRS complexes based on morphology and rhythm to estimate where they originate.

However, these algorithms can be problematic. For example, they may not classify QRS complexes at all, or may classify QRS complexes incorrectly, which increases the burden on clinical staff and can result in alert fatigue.

### SUMMARY OF THE DISCLOSURE

There is thus a continued need for methods and systems that efficiently and accurately analyze ECG signals to correctly classify QRS complexes.

Various embodiments and implementations are directed to a method and system for automated ECG signal classification. An ECG analysis system receives an ECG signal classification for a subject, comprising an ECG signal and a plurality of identified QRS complexes in the ECG signal. The system analyzes the ECG signal classification to identify at least one erroneous or dissimilar identified QRS complex and generate a final ECG signal classification. The analysis includes filtering the received ECG signal, extracting a plurality of features from each of the plurality of identified QRS complexes; clustering the identified QRS complexes into at least a first cluster of QRS complexes and a cluster of abnormal or dissimilar QRS complexes; generating a QRS cluster template from the first cluster of QRS complexes; calculating a distance between each of the QRS complexes in the cluster of abnormal or dissimilar QRS complexes and the QRS cluster template; and classifying, based on the calculated distance, the QRS complexes in the cluster of abnormal or dissimilar QRS complexes as being normal or abnormal/dissimilar to generate the final ECG signal classification. The generated final ECG signal classification is then reported.

According to an aspect, a method for automated analysis of ECG signal classifications is provided. The method includes receiving an ECG signal classification for a subject, comprising an ECG signal and a plurality of identified QRS complexes in the ECG signal, wherein at least one of the identified plurality of identified QRS complexes is an erroneous identification or dissimilar from other QRS complexes in the ECG signal; analyzing the ECG signal classification to identify the at least one erroneous or dissimilar identified QRS complex and generate a final ECG signal classification, comprising: (i) filtering the received ECG signal; (ii) extracting a plurality of features from each of the plurality of identified QRS complexes; (iii) clustering, using the extracted plurality of features, the identified QRS complexes into at least a first cluster of QRS complexes and a cluster of abnormal or dissimilar QRS complexes; (iv) generating a QRS cluster template from the first cluster of QRS complexes; (v) calculating a distance between each of the QRS complexes in the cluster of abnormal or dissimilar QRS complexes and the QRS cluster template; and (vi) classifying, based on the calculated distance, the QRS complexes in the cluster of abnormal or dissimilar QRS complexes as being normal or abnormal/dissimilar to generate the final ECG signal classification; and reporting the generated final ECG signal classification.

According to an embodiment, the method further includes identifying, using a QRS complex detection algorithm, a plurality of QRS complexes in an ECG signal.

According to an embodiment, filtering the received ECG signal comprises removing baseline wander.

According to an embodiment, filtering the received ECG signal comprises bandpass cascaded filtering.

According to an embodiment, analyzing the ECG signal classification further comprises reducing dimensionality of extracted features.

According to an embodiment, clustering the identified QRS complexes comprises Gaussian mixture modeling and/or hierarchical clustering.

According to an embodiment, the method further includes analyzing the generated final ECG signal classification to generate a diagnosis of the patient; and administering, based on the diagnosis of the patient, a treatment to the patient configured to address the diagnosis.

According to an embodiment, the treatment is one or more of an arrhythmia treatment medication, cardioversion, ablation, a pacemaker, or other treatment.

According to another aspect is a system for automated analysis of ECG signal classifications. The system includes an ECG signal classification for a subject, comprising an ECG signal and a plurality of identified QRS complexes in the ECG signal, wherein at least one of the identified plurality of identified QRS complexes is an erroneous identification or dissimilar from other QRS complexes in the ECG signal; a processor configured to analyze the ECG signal classification to identify the at least one erroneous or dissimilar identified QRS complex and generate a final ECG signal classification, wherein the processor is configured to: (i) filter the received ECG signal; (ii) extract a plurality of features from each of the plurality of identified QRS complexes; (iii) cluster, using the extracted plurality of features, the identified QRS complexes into at least a first cluster of QRS complexes and a cluster of abnormal or dissimilar QRS complexes; (iv) generate a QRS cluster template from the first cluster of QRS complexes; (v) calculate a distance between each of the QRS complexes in the cluster of abnormal or dissimilar QRS complexes and the QRS cluster template; and (vi) classify, based on the calculated distance, the QRS complexes in the cluster of abnormal or dissimilar QRS complexes as being normal or abnormal/dissimilar to generate the final ECG signal classification; and a user interface configured to provide the generated final ECG signal classification.

According to an embodiment, the system further includes a diagnosis of the patient based on an analysis of the generated final ECG signal classification; and a treatment administered to the patient and based on the diagnosis of the patient, wherein the treatment is one or more of an arrhythmia treatment medication, cardioversion, ablation, a pacemaker, or other treatment.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for automated analysis of ECG signal classifications, in accordance with an embodiment.

FIG. 2 is a schematic representation of an ECG analysis system, in accordance with an embodiment.

FIG. 3 is a flowchart of a method for automated analysis of ECG signal classifications, in accordance with an embodiment.

FIG. 4 is a flowchart of a method for baseline wander filtering, in accordance with an embodiment.

FIG. 5 a flowchart of a method for FIR filtering, in accordance with an embodiment.

FIG. 6 is a flowchart of a method for energy signal calculation, in accordance with an embodiment.

FIG. 7 is a schematic representation of a QRS complex window, in accordance with an embodiment.

FIG. 8 is a flowchart of a method for QRS complex width calculation process, in accordance with an embodiment.

FIG. 9 is a flowchart of a method for adaptive feature selection, in accordance with an embodiment.

FIG. 10 is a flowchart of a method for classification flow, in accordance with an embodiment.

FIG. 11 is a flowchart of a method for calculating Euclidian distance path, in accordance with an embodiment.

FIG. 12 a flowchart of a method for QRS complex distance comparison, in accordance with an embodiment.

### DETAILED DESCRIPTION OF EMBODIMENTS

The present disclosure describes various embodiments of a system and method configured to generate and provide QRS complex classifications using an automated ECG analysis system. More generally, Applicant has recognized and appreciated that it would be beneficial to more efficiently and accurately identify and classify QRS complexes in an ECG signal, thereby reducing clinician burden. In addition, this method can be leveraged to distinguish dissimilar populations of QRS complexes. Accordingly, the ECG analysis system receives an ECG signal classification for a subject, comprising an ECG signal and a plurality of identified QRS complexes in the ECG signal. The system analyzes the ECG signal classification to identify at least one erroneous identified QRS complex and generate a final ECG signal classification. The analysis includes filtering the received ECG signal, extracting a plurality of features from each of the plurality of identified QRS complexes; clustering the identified QRS complexes into at least a first cluster of QRS complexes and a second cluster of dissimilar or abnormal QRS complexes; generating a QRS cluster template based on the population with the greatest number; calculating a distance between each of the QRS complexes in the cluster of abnormal or dissimilar QRS complexes and the QRS cluster template; and classifying, based on the calculated distance, the QRS complexes in the cluster of abnormal or dissimilar QRS complexes as being similar to the general population of QRS complexes. This can then be used to determine if that population is normal or abnormal/dissimilar, generating the final ECG signal classification. The generated final ECG signal classification is then reported.

According to an embodiment, the methods and systems described or otherwise envisioned herein can be utilized to, for example, identify false positive or false negative QRS complex classifications determined by existing algorithms using unsupervised methods. In addition, it can cluster QRS complexes into multiple populations without an underline classification. These issues are commonly known and are often reported by clinical staff causing increased review burden and alert fatigue. According to an embodiment, an ECG system identifies dissimilar populations of QRS complexes, false positive or false negative QRS complex classifications, and/or clusters QRS complexes based on similar features.

Thus, according to an embodiment, the methods and systems described or otherwise envisioned herein can also be utilized to distinguish dissimilar populations of QRS complexes, among other uses.

The embodiments and implementations disclosed or otherwise envisioned herein can be utilized with any ECG system or process that may utilize or benefit from improved QRS complex classification. For example, the systems and methods described or otherwise envisioned herein can, in some non-limiting embodiments, be implemented as an element for a commercial product for patient analysis or monitoring, such as Philips® ECG systems and devices (available from Koninklijke Philips NV, the Netherlands), or any other suitable system. However, the disclosure is not limited to these devices or systems, and thus disclosure and embodiments disclosed herein can encompass any system that may utilize or benefit from improved QRS complex classification.

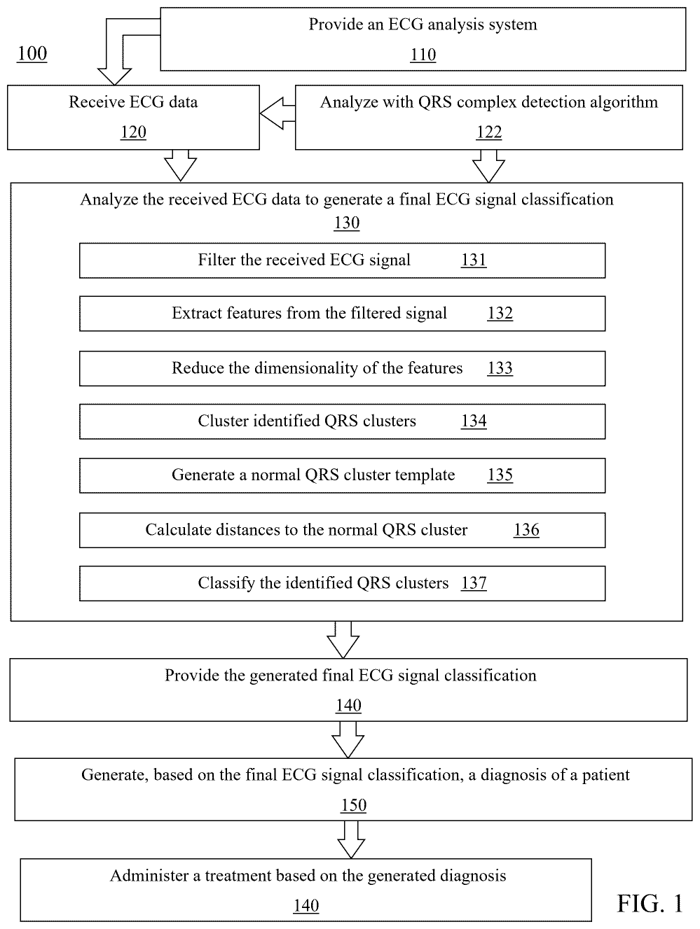

Referring to FIG. 1, in one embodiment, is a flowchart of a method 100 for automated analysis of ECG signal classifications using an ECG analysis system 200. The methods described in connection with the figures are provided as examples only, and shall be understood not to limit the scope of the disclosure. The ECG analysis system can be any of the devices or systems described or otherwise envisioned herein. The ECG analysis system can be a single device or system, or can be multiple different devices or systems.

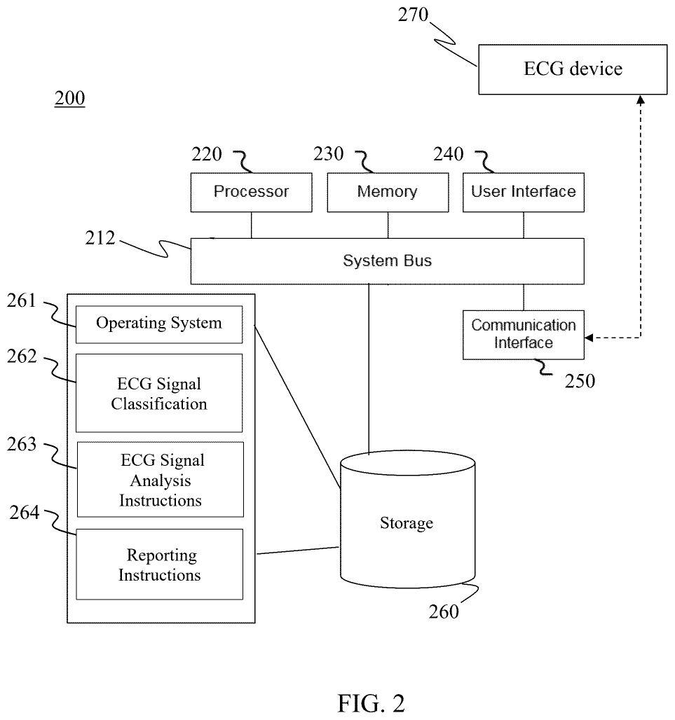

At step 110 of the method, the ECG analysis system 200 is provided. Referring to an embodiment of ECG analysis system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the ECG analysis system 200 may be different and more complex than illustrated. Additionally, ECG analysis system 200 can be any of the devices described or otherwise envisioned herein. Other elements and components of the ECG analysis system 200 are disclosed and/or envisioned elsewhere herein.

According to an embodiment, the ECG analysis system 200 comprises or is in direct or indirect communication with an ECG device 270 configured to obtain ECG data from a subject. The ECG device 270 can be any ECG device, including devices known in the art. The ECG device comprises one or more ECG leads positioned in contact with a subject, according to known methods, and can obtain ECG data from that subject. According to another embodiment, the ECG analysis system 200 comprises or is in direct or indirect communication with a server, database, or other device comprising recorded ECG data. Once obtained, the ECG data may be utilized immediately, and/or it may be temporarily or permanently stored in memory for future use.

At step 120 of the method, the ECG analysis system 200 receives, requests, or otherwise obtains ECG data comprising an ECG signal classification for the subject, comprising an ECG signal and a plurality of identified QRS complexes in the ECG signal. The system may obtain an ECG signal using known methods for obtaining ECG signals, which may be a recorded signal. The signal may be obtained continuously or periodically as determined by the settings or parameters of the ECG device or system. Once obtained, the ECG signal may be utilized immediately and/or it may be saved in memory for future analysis.

According to an embodiment, the ECG signal classification comprises both the ECG signal and a plurality of QRS complexes identified in the ECG signal. The plurality of QRS complexes may be identified, for example, manually and/or using a QRS complex classification algorithm, among other methods.

According to an embodiment, some of the plurality of identified or classified QRS complexes in the ECG signal are misidentified or misclassified. For example, the QRS classifications may comprise false negatives in which abnormal or dissimilar QRS complexes are classified as normal QRS complexes, and/or false positives in which normal QRS complexes are classified as abnormal or dissimilar QRS complexes. Other misidentifications or misclassifications are possible.

At optional step 122, which can occur at any point prior to step 130, a QRS complex detection algorithm can analyze an ECG signal to identify a plurality of QRS complexes in the ECG signal. The QRS complex detection algorithm can be any algorithm known in the art or developed to perform QRS complex detection. According to an embodiment, the system receives an ECG signal and performs the QRS complex detection using the detection algorithm. According to another embodiment, the system receives an ECG signal for which QRS complex detection has already been performed.

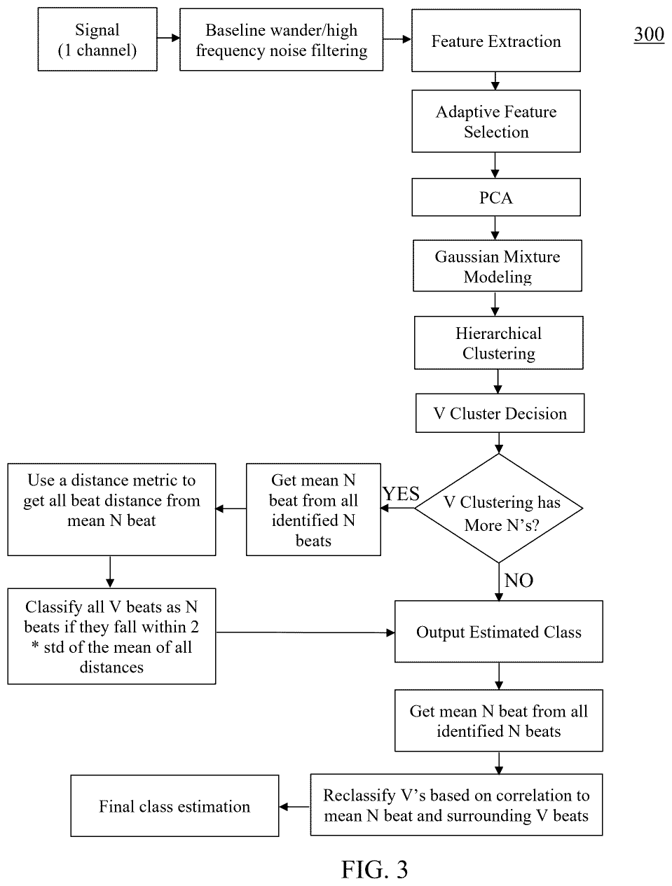

At step 130 of the method, the ECG system analyzes the ECG signal classification to identify false positives, false negatives, or other QRS complex misidentifications or misclassifications. The analysis results in a final ECG signal classification that can be provided to a clinician for review and patient diagnosis or can be provided to another device or system for downstream use such as a monitoring system. Thus, the analysis by the ECG analysis system can improve diagnoses and reduce false alarms, among other improvements. Referring to FIG. 3, in one embodiment, is a flowchart 300 of the ECG analysis method.

The analysis of the ECG signal classification by the ECG analysis system is a multi-step unsupervised process. At step 131 of the analysis, the ECG system filters the received ECG signal. The filtering can comprise one or multiple steps and can be a variety of different possible filters. According to an embodiment, the filtering comprises removing both baseline wander and high frequency noise.

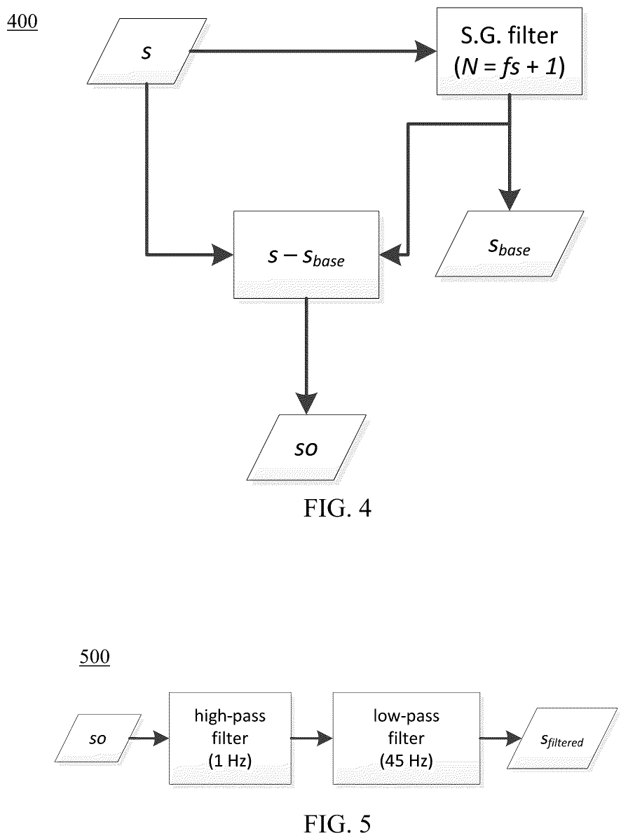

According to an embodiment, filtering can comprise a Savitzky-Golay (S.G.) filter, although other filters are possible. The S.G. filter is used to remove baseline wander. First, a copy of the signal (s_{base}) is passed through the S.G. filter with a window length of fs+1 with a polynomial order of 3. The increased window length dramatically smooths out the s_{base} so that only the underlying baseline wander remains. s_{base} is subtracted from the original signal(s), resulting in a signal so that minimizes the distortion to the P-QRS-ST cycle. Referring to FIG. 4, in one embodiment, is a flowchart 400 of the baseline wander filtering.

According to an embodiment, filtering can additionally or alternatively comprise one or more finite impulse response (FIR) filters. For example, filtering can comprise two cascaded finite impulse response filters used to bandpass the signal between 1 to 45 Hz. Referring to FIG. 5, in one embodiment, is a flowchart 500 of the FIR filtering. FIR filtering removes remaining baseline wander along with high frequency noise, including power line interference. The first filter has fs+1 taps and a highpass cutoff of 1 Hz. The second is a low-pass Butterworth filter with 6 taps and a cutoff frequency of 45 Hz. Both are implemented using the FIR window method with a Hamming window. Since baseline wander can often mimics premature ventricular contraction (PVC) characteristics, the high-pass filter has a large coefficient number to remaining bandpass wander leakage. The low number of taps in the Butterworth filter is chosen to not affect ST level measurements. The coefficients for the filters will vary depending on the sampling frequency of the signal and are not listed here.

At step 132 of the analysis, the ECG system extracts a plurality of features from each of the plurality of identified QRS complexes. Feature extraction can be performed in a wide variety of ways, and can include one or multiple extracted features.

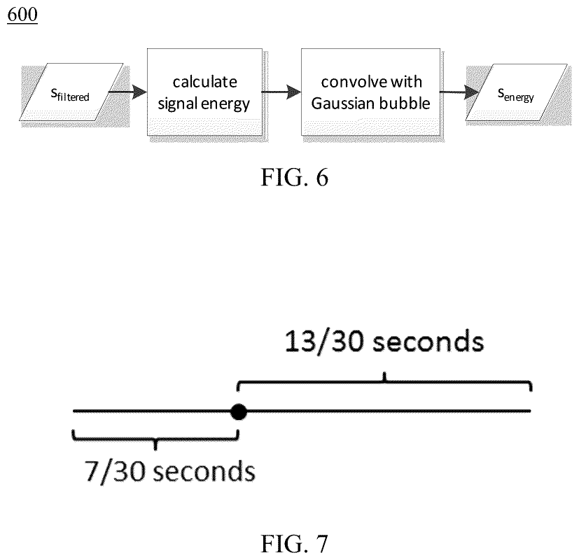

According to an embodiment, for feature extraction a search window is created around each QRS complex detected by a QRS complex detection algorithm for feature extraction. The search window should have a total width of ⅔ seconds, starting 7/30 seconds before the QRS complex and ending 13/30 seconds after the QRS complex, as shown for example in FIG. 7. The features can then be extracted.

A wide variety of features can be extracted and utilized, from one feature to a plurality of features. Below, a total of seventeen (17) features are utilized and found within the search window for each QRS complex. However, this is a non-limiting example and fewer, additional, and/or other features may be utilized.

According to an embodiment, filtering comprises a generalized Teager-Kaiser energy operator (GTEO). The generalized Teager-Kaiser energy operator (GTEO) can be defined as follows:

s_{energy,m}[n]=x²[n]−x[n−m]x[n+m] (Eq. 1)

According to an embodiment, it generates a time series that represents the local energy of the ECG signal. Changing the m parameter corresponds to calculating the instantaneous energy in different frequency bands. m is chosen to be 7 based on literature values. For the boundary conditions where n−m<0 or n+m>num_samples, take x[n−m] and x[n+m] to be 0. To better emphasize the peaks, the output of the GTEO can be convolved with a Gaussian window. The Gaussian window is 0.15 seconds×fₛ samples long with a standard deviation of 10. Referring to FIG. 6, in one embodiment, is a flowchart 600 of the flow of the energy signal calculation.

### Feature 1—QRS Complex Maximum Energy

According to an embodiment, the QRS complex maximum energy is taken from the GTEO results. For each QRS complex's corresponding search window, the maximum GTEO value (max{s_{energy,m}[n]}) is found. Since the GTEO follows the envelope of the QRS peak, the maximum point will indicate whether a QRS complex's envelope is depressed or elevated when comparing to other QRS complexes.

### Feature 2—QRS Complex Integrated Energy

The QRS complex integrated energy is taken from the GTEO results. For each QRS complex's corresponding search window, a trapezoidal integration of the GTEO signal is performed. Since a QRS complex isn't localized to a single point, this feature gives information about how the energy spread across the QRS complex.

### Feature 3—Maximum Local Derivative

The maximum local derivative is taken from the filtered signal. Within the search window, a difference is performed:

dx[n]=x[n]−x[n−1] (Eq. 2)

The maximum value of the differenced signal (max{dx[n]}) is computed, which helps identify how fast a QRS slope is rising.

### Feature 4—Minimum Local Derivative

The minimum local derivative is taken from the filtered signal. Within the search window, a difference is performed:

dx[n]=x[n]−x[n−1] (Eq. 3)

The minimum value of the differenced signal (min {dx[n]}) is computed, which helps distinguish how fast a QRS slope is decreasing, particularly in cases where the PVCs results in slow-varying, deep S-waves.

### Feature 5—Signal Maximum

The signal maximum is taken from the filtered signal (max{sₒ[n]}), which helps identify QRS complexes with tall R-waves.

### Feature 6—Signal Minimum

The signal minimum is taken from the filtered signal (min {sₒ[n]}), which helps separate depressed QRS complex S-waves.

### Feature 7—Local Maxima-Minima Difference (LMMD)

The local maxima-minima difference (LMMD) is taken from the filtered signal. It is calculated with the following:

LMMD=max{sₒ[n]}−min{sₒ[n]} (Eq. 4)

It is expected that for a PVCs, the local peak and valley to exhibit a larger LMMD than a normal QRS complex.

### Feature 8—QRS Complex Width

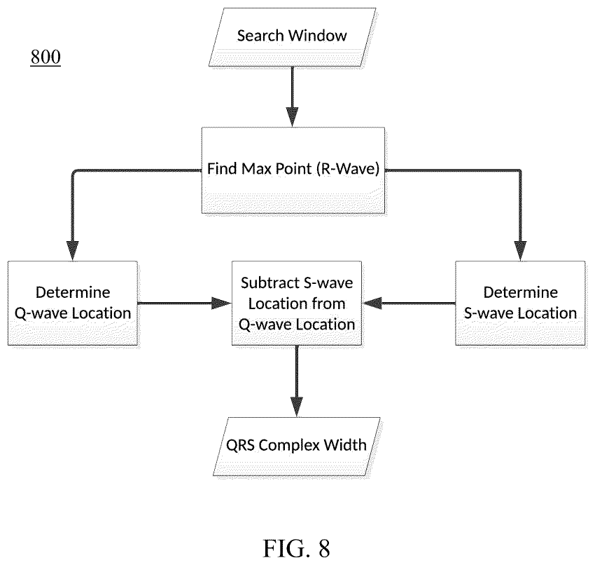

The QRS complex width is taken from the filtered signal. It is the relative temporal width of the QRS complex from the Q-wave to the S-wave. First, the index of the of the maximum point in the search window is determined. To find the Q-wave minimum, move backwards from the maximum point, stopping where the derivative of the previous point is less than zero and the derivative of the current point is greater than zero. To find the S-wave minimum, move forwards from the maximum point, stopping where the derivative of the current point is greater than zero and the derivative of the next point is less than zero. The difference between the S-wave and Q-wave minimums found is the QRS complex width. Referring to FIG. 8 is a flowchart 800 of the QRS complex width calculation process.

### Feature 9—Area Above QRS Complex Isoelectric Point

The area above the QRS complex isoelectric point is taken from the filtered signal. Once the QRS complex width is found, the absolute value of the area between the Q-wave minimum and the maximum point is integrated. There usually is a difference in area above the QRS complex isoelectric point when comparing normal and abnormal QRS complexes.

### Feature 10—Area Below QRS Complex Isoelectric Point

The area below the QRS complex isoelectric point is taken from the filtered signal. Once the QRS complex width is found, the absolute value of the area between the maximum point and the S-wave minimum is integrated. There usually is a difference in area below the QRS complex isoelectric point when comparing normal and abnormal QRS complexes.

### Feature 11—Max Spectral Amplitude (MSA)

The max spectral amplitude (MSA) is taken from the Fourier transform of the filtered signal. First, a periodogram is calculated from the search window using NFFT=5×fs. Then, return the values in the periodogram within the frequency range of 2 to 5 Hz. The maximum value returned is the MSA. This will help target QRS complexes in the low frequency range.

### Feature 12—Spectral Amplitude Sum

The spectral amplitude sum is taken from the Fourier transform of the filtered signal. First, calculate a periodogram from the search window using NFFT=5×fs. Then, the values in the periodogram within the frequency range of 2 to 5 Hz are returned. Finally, the returned values are summed. Since a QRS complex isn't localized to a single frequency, this feature gives information about how the QRS complex energy is spread across these frequencies.

### Feature 13—Entropy

Entropy is taken from the Fourier transform of the filtered signal. First, a periodogram is calculated from the search window using NFFT=5×fs. The values in the periodogram are divided by the sum of the periodogram to get Pxxᵢ. Next, the entropy is obtained with the following equation:

entropy=−ΣᵢPxxᵢ ln(Pxxᵢ) (Eq. 5)

A small value (1e-10) is added to the quotient to prevent the logarithm result from becoming a NaN value. The entropy feature estimates how concentrated the frequency content is.

### Feature 14—Kurtosis

Kurtosis is taken from the filtered signal. It is a measure of how skewed a distribution is. Taking the signal within the search window as a distribution, this feature can differentiate normal and abnormal QRS complexes based on how skewed the QRS complex is from the R-wave.

### Feature 15—Autoregressive Coefficients

Autoregressive (AR) coefficients are taken from the filtered signal. The first two coefficients are estimated through the Burg method. Previous work has demonstrated that the AR coefficients can be a good discriminator between normal and abnormal QRS complexes.

### Feature 16—Inversion

The Invert feature aims at quantifying whether the amplitude of the R-wave or S-wave is greater. This is determined by comparing the absolute values of R-wave and S-wave. If the R-wave is greater than the S-wave, the amplitude of the R-wave is stored as Invert. If the S-wave is greater than the R-wave, the negative amplitude is stored as Invert.

### Feature 17—RR-Interval Ratio

Abnormal QRS complexes can differ in prematurity compared to the former QRS complex and have a compensatory pause compared to the next QRS complex. These characteristics of premature QRS complexes are exploited in the RR-interval ratio. The ratio is calculated as follows:

$$R {R}_{ratio} = \frac{R {R}_{next}}{R {R}_{prev}} \qquad ( Eq . 6 )$$

RRₙₑₓₜ represents the time difference between the next QRS complex and the current QRS complex, while RRₚᵣₑᵥ represents the time difference between the current QRS complex and the previous QRS complex. For normal QRS complexes, RRₙₑₓₜ should approximately be equal to RRₚᵣₑᵥ (RRᵣₐₜᵢₒ≈1). For premature QRS complexes without a noticeable compensatory pause, the denominator will be smaller, making the ratio greater than one. For premature QRS complexes with a noticeable compensatory pause, the numerator will be larger while the denominator will be smaller, causing the ratio to be much greater than one. This information is summarized in TABLE 1.

TABLE 1
RR Ratio Expected Values

| | ratio |
|---|---|
| Normal QRS complex | ≈1 |
| QRS complex with prematurity | >1 |
| QRS complex with prematurity and compensatory pause | >>1 |

### Adaptive Feature Selection.

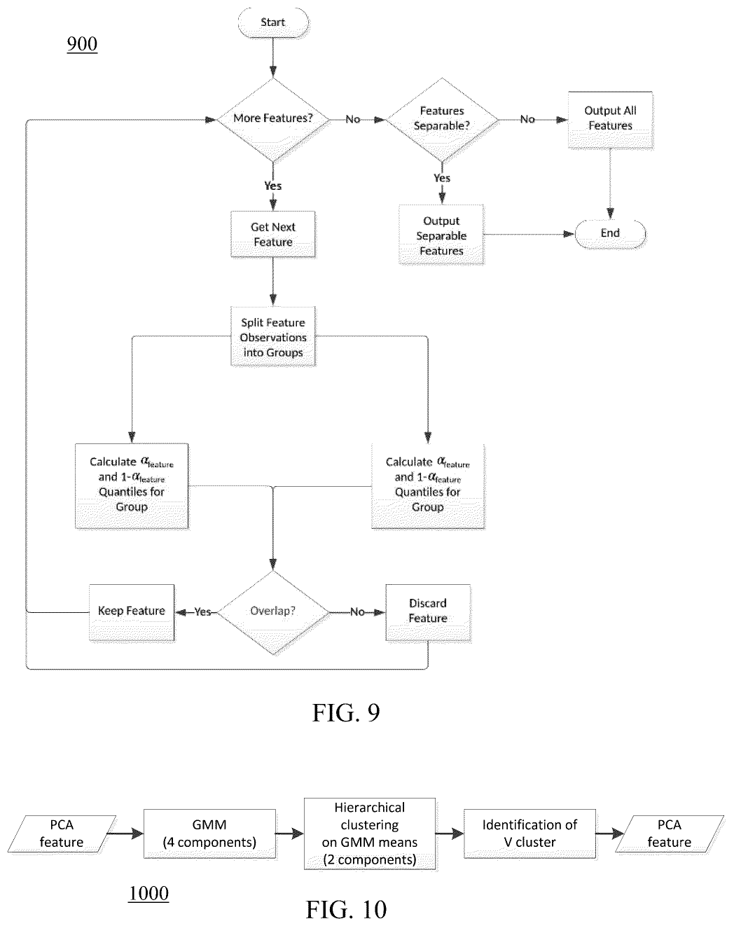

Since every patient has a unique physiology, some features will be more relevant to a particular ECG strip than others. Retaining irrelevant features only obfuscates decision boundaries in classification. Adaptive feature selection is implemented to choose features with the greatest separation between normal and abnormal QRS complexes. According to an embodiment, adaptive feature selection is achieved by comparing separation between the normal and abnormal QRS complex distributions for a particular feature. α_{feature}/2 and 1—α_{feature}/2 quantiles are calculated for each distribution. If the quantiles do not overlap between the distributions, then the feature is kept. If they do, then the feature is discarded. If less than two of the features are separable, then all the features are kept. Referring to FIG. 9 is a flowchart 900 of the adaptive feature selection. The α_{feature} value is chosen to be 0.3 based on Sp and PPV values for normal and PVC QRS complexes classification after testing on the MIT/AHA data.

The adaptive feature selection was compared to pre-selected features that demonstrated the most influence on PVC QRS complex discrimination throughout the MIT/AHA datasets. The latter features were determined by performing a LASSO logistic regression for PVC QRS complexes using all 17 features. The three features that were found to be significant in over 50% of the datasets were: (1) entropy, (2) kurtosis, and (3) RR-interval ratio. The results demonstrate that adaptive feature selection gives a much higher positive predictability than pre-selected features alone.

At step 133 of the method, which can be optional, the ECG system reduces the dimensionality of the extracted one or more features. Dimensionality reduction can be done in a wide variety of ways. According to one possible embodiment, dimensionality reduction is done by principal component analysis (PCA).

According to an embodiment, PCA performs further dimensionality reduction by mixing the chosen features into n hybrid features that maximizes the separation between identified normal and abnormal QRS complexes. From testing performed on the MIT/AHA datasets comparing normal and PVCs, n was chosen to be 1.

At step 134 of the method, the ECG system clusters the identified QRS complexes using the extracted plurality of features. The identified QRS complexes can be clustered in a variety of ways, using any of a plurality of possible mechanisms. According to an embodiment, the identified QRS complexes are clustered into at least a first cluster of QRS complexes and a cluster of abnormal or dissimilar QRS complexes, although other and/or more clusters are possible.

According to an embodiment, unsupervised clustering is used to distinguish QRS complexes with different morphology (e.g., “normal” and “abnormal” or “dissimilar” QRS complexes) using the features generated from PCA. The first step is to perform initial classification using Gaussian mixture models. Full covariance matrices are chosen to maximize the volume that a Gaussian mixture can cover. The number of Gaussian mixtures can be chosen based on testing using the MIT/AHA datasets comparing normal and PVCs.

According to an embodiment, the number of Gaussian mixtures which gives the best balance of high sensitivity, specificity, and positive predictability is 4. Multiple Gaussian mixtures help capture some of the variance in the nonlinear PCA distribution.

In the case where one wants to separate the QRS complexes into two groups (e.g., normal and PVC QRS complexes), the Gaussian mixtures are combined through hierarchical clustering on the Gaussian mixture means using single linkage, combining clusters whose means have shortest distances into super clusters. Single linkage combines clusters with the closest elements and is used under the assumption that the only difference between two clusters in proximity is noise. Hierarchical clustering stops once there are only two clusters remaining. If the goal is to include more than two clusters or the output of the GMM clustering contains only two unique clusters, the hierarchical clustering step is skipped. In the original implementation to separate normal and PVC complexes, the cluster with the most PVCs identified was chosen to be the PVC cluster and all normal QRS complexes identified were retained. Referring to FIG. 10, in accordance with an embodiment, is a flowchart 1000 of the classification flow.

At step 135 of the method, the ECG system generates a QRS cluster template from the clustered ECG complexes. The QRS cluster template can be generated in a variety of different ways. According to an embodiment, this QRS cluster template is generated for so-called “normal” QRS complexes, e.g., the first QRS cluster, and according to at least one embodiment, comprises the cluster with the most QRS complexes.

According to an embodiment, if there were more so-called “normal” QRS complexes than PVC QRS complexes in the PVC QRS complex hierarchical cluster then the process goes down a new path for PVC QRS complex identification. The ratio threshold for detected normal and PVC complexes in the PVC cluster was determined through testing on the MIT/AHA datasets.

For each QRS complex, a signal segment starting 0.15 s before the QRS complex and ending 0.15 s after the QRS complex is extracted. If the QRS complex is too close to the beginning or end of the ECG recording, the segment is padded with 0's so that its length is equal to 0.30 s and the R-wave is centered. According to an embodiment, all the identified so-called “normal” QRS complex segments across each time point are averaged to get a normal QRS complex template. Using Euclidean distance, the distance between the normal QRS complex template and the individual normal QRS complex segments is calculated. The mean and standard deviation of resultant distance distribution is determined.

At step 136 of the method, a distance between each of the QRS complexes in the cluster of abnormal or dissimilar QRS complexes and the QRS cluster template generated from the first QRS cluster is calculated. This distance can be calculated in a variety of ways.

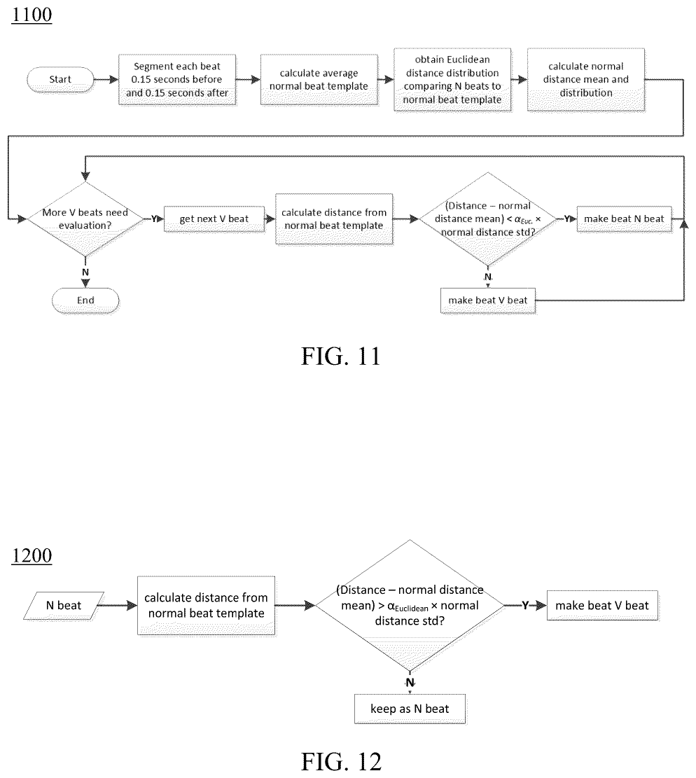

According to an embodiment, the distance between each identified PVC segment and the QRS complex template is calculated. If the difference between this distance and the mean of the QRS complex distances is less than α_{Dist} multiplied by the QRS complex distance standard deviation, then this QRS complex is changed to a first or “normal” QRS complex. α_{Dist} is chosen to be two (2) in order to minimize false negative PVCs. All identified normal QRS complexes are retained as normal QRS complexes. Referring to FIG. 11 is a flowchart 1100 of this process of calculating the Euclidian distance path.

At step 137 of the method, the ECG system classifies, based on the calculated distance, the QRS complexes in the cluster of dissimilar or abnormal QRS complexes as being normal or dissimilar or abnormal to generate the final ECG signal classification. According to an embodiment, this comprises correcting one or more false negative QRS complexes in the cluster of normal QRS complexes to a dissimilar or abnormal QRS complex

According to an embodiment, when trying to separate normal and PVC QRS complexes, the clustering described herein can group PVCs with the majority of the normal QRS complexes due to multifocal PVCs or poor signal quality. This can be commonly seen when a PVC may only have marginally greater amplitude than surrounding normal QRS complexes or when a PVC is corrupted by noise. The following corrects these false negative PVCs by checking for normal QRS complex conformity with specific patterns of PVC runs.

According to an embodiment, for each QRS complex detected, a search window is created. The search window should have a total width of ⅔ seconds, starting 7/30 seconds before the QRS complex and ending 13/30 seconds after the QRS complex, as described above. If the QRS complex is too close to the beginning or end of the strip, pad the search window with 0's so that the search window length is equal to ⅔ seconds.

According to an embodiment, all identified normal QRS complexes are averaged across each time point to get a normal QRS complex template. The Euclidean distance between the normal QRS complex template and the individual normal QRS complex windows (Template_{distance}) is calculated. The mean and standard deviation of resultant distance distribution is determined. Next, the following QRS complex pattern runs are identified: (1) NVVVN, (2) NVNVN, (3) NVNNV, (4) NVNVV, (5) NVVNV, (6) VNNVV, (7) VVNNV, (8) VVNVV, where N and V represent normal and PVC QRS complexes. Descriptions for how to correct for PVCs in the pattern runs are given below. Generally, normal QRS complexes are correlated with surrounding PVCs. If the normal QRS complex is positively correlated with the majority of the surrounding PVCs, it is considered a PVC complex with poor signal quality. If a normal QRS complex is not well correlated with its surrounding PVCs, its Euclidean distance from the normal QRS complex template is taken. Euclidean distance comparisons described in the next subsections are similar to the DTW ones and outlined in FIG. 12, which is a flowchart 1200 of the QRS complex distance comparison.

According to an embodiment, α_{Euclidean} is equal to two (2), and all identified normal QRS complexes are retained.

### QRS Complex Pattern 1—NVVVN Run

According to an embodiment, correlations are calculated between:
QRS complex 1 (N) and QRS complex 2 (V)
QRS complex 1 (N) and QRS complex 3 (V)
QRS complex 1 (N) and QRS complex 4 (V)
QRS complex 5 (N) and QRS complex 2 (V)
QRS complex 5 (N) and QRS complex 3 (V)
QRS complex 5 (N) and QRS complex 4 (V)

If at least two of the correlations between QRS complex 1 and QRS complexes 2, 3, or 4 are greater than 0, QRS complex 1 is changed into a PVC. If at least two of the correlations between QRS complex 5 and QRS complexes 2, 3, or 4 are greater than 0, QRS complex 5 is changed into a PVC. This corrects for QRS complexes that are oriented in the same direction as identified PVCs but are being misidentified as normal QRS complexes due to poor signal quality.

Next, for QRS complex 1 and QRS complex 5, the distance is calculated between each QRS complex's search window and the mean normal QRS complex window. If the difference between this distance and the mean of the normal QRS complex distances is greater than α_{Euclidean} multiplied by the normal QRS complex distance standard deviation, then this QRS complex is changed to a PVC. This corrects for multifocal PVCs.

### QRS Complex Pattern 2—NVNVN Run

According to an embodiment, correlations are calculated between:
QRS complex 3 (N) and QRS complex 2 (V)
QRS complex 3 (N) and QRS complex 4 (V)

If both correlations between QRS complex 3 and QRS complexes 2 and 4 are greater than 0, QRS complex 3 is changed into a PVC. Next the correlation between (where V′ is the N to V converted QRS complex) is calculated:
QRS complex 5 (N) and QRS complex 3 (V′)
QRS complex 5 (N) and QRS complex 2 (V)
QRS complex 5 (N) and QRS complex 4 (V)

If all three QRS complexes correlate with QRS complex 5, then QRS complex 5 is changed into a PVC. Then, the correlation between the following is calculated:
QRS complex 1 (N) and QRS complex 3 (V′)
QRS complex 1 (N) and QRS complex 2 (V)
QRS complex 1 (N) and QRS complex 4 (V)

If all three QRS complexes correlate with QRS complex 1, then QRS complex 1 is changed into a PVC. This corrects for QRS complexes that are oriented in the same direction as identified PVCs but are being misidentified as normal QRS complexes due to poor signal quality.

### QRS Complex Pattern 3—NVNNV Run

According to an embodiment, correlations are calculated between:
QRS complex 3 (N) and QRS complex 2 (V)
QRS complex 3 (N) and QRS complex 5 (V)

If both correlations between QRS complex 3 and QRS complexes 2 and 5 are greater than 0, QRS complex 3 is changed into a PVC. Next, the correlation between (where V′ is the N to V converted QRS complex) is calculated:
QRS complex 4 (N) and QRS complex 3 (V′)
QRS complex 4 (N) and QRS complex 2 (V)
QRS complex 4 (N) and QRS complex 5 (V)

If all three QRS complexes correlate with QRS complex 4, then QRS complex 4 is changed into a PVC. Then, the correlation between (where V′ is the N to V converted QRS complex) is calculated:
QRS complex 1 (N) and QRS complex 3 (V′)
QRS complex 1 (N) and QRS complex 2 (V)
QRS complex 1 (N) and QRS complex 4 (V)

If all three QRS complexes correlate with QRS complex 1, then QRS complex 1 is changed into a PVC. This corrects for QRS complexes that are oriented in the same direction as identified PVCs but are being misidentified as normal QRS complexes due to poor signal quality.

### QRS Complex Pattern 4—NVNVV Run

According to an embodiment, correlations are calculated between:
QRS complex 3 (N) and QRS complex 2 (V)
QRS complex 3 (N) and QRS complex 4 (V)
QRS complex 3 (N) and QRS complex 5 (V)

If at least two of the correlations between QRS complex 3 and QRS complexes 2, 4, or 5 are greater than 0, QRS complex 3 is changed into a PVC. This corrects for QRS complexes that are oriented in the same direction as identified PVCs but are being misidentified as normal QRS complexes due to poor signal quality.

### QRS Complex Pattern 5—NVVNV Run

According to an embodiment, correlations are calculated between:
QRS complex 4 (N) and QRS complex 2 (V)
QRS complex 4 (N) and QRS complex 3 (V)
QRS complex 4 (N) and QRS complex 5 (V)
QRS complex 1 (N) and QRS complex 2 (V)
QRS complex 1 (N) and QRS complex 3 (V)
QRS complex 1 (N) and QRS complex 5 (V)

If at least two of the correlations between QRS complex 4 and QRS complexes 2, 3, or 5 are greater than 0, QRS complex 4 is changed into a PVC. If at least two of the correlations between QRS complex 1 and QRS complexes 2, 3, or 5 are greater than 0, QRS complex 1 is changed into a PVC. This corrects for QRS complexes that are oriented in the same direction as identified PVCs but are being misidentified as normal QRS complexes due to poor signal quality.

### QRS Complex Pattern 6—VNNVV Run

According to an embodiment, correlations are calculated between:
QRS complex 2 (N) and QRS complex 1 (V)
QRS complex 2 (N) and QRS complex 4 (V)
QRS complex 2 (N) and QRS complex 5 (V)
QRS complex 3 (N) and QRS complex 1 (V)
QRS complex 3 (N) and QRS complex 4 (V)
QRS complex 3 (N) and QRS complex 5 (V)

If at least two of the correlations between QRS complex 2 and QRS complexes 1, 4, or 5 are greater than 0, QRS complex 2 is changed into a PVC. If at least two of the correlations between QRS complex 3 and QRS complexes 1, 4, or 5 are greater than 0, QRS complex 3 is changed into a PVC. This corrects for QRS complexes that are oriented in the same direction as identified PVCs but are being misidentified as normal QRS complexes due to poor signal quality.

Next, for QRS complex 2 and QRS complex 3, the distance is calculated between each QRS complex's search window and the mean normal QRS complex window. If the difference between this distance and the mean of the normal QRS complex distances is greater than Euclidean multiplied by the normal QRS complex distance standard deviation, then this QRS complex is changed to a PVC. This corrects for multifocal PVCs.

### QRS Complex Pattern 7—VVNNV Run

According to an embodiment, correlations are calculated between:
QRS complex 3 (N) and QRS complex 1 (V)
QRS complex 3 (N) and QRS complex 2 (V)
QRS complex 3 (N) and QRS complex 5 (V)
QRS complex 4 (N) and QRS complex 1 (V)
QRS complex 4 (N) and QRS complex 2 (V)
QRS complex 4 (N) and QRS complex 5 (V)

If at least two of the correlations between QRS complex 3 and QRS complexes 1, 2, or 5 are greater than 0, QRS complex 3 is changed into a PVC. If at least two of the correlations between QRS complex 4 and QRS complexes 1, 2, or 5 are greater than 0, QRS complex 4 is changed into a PVC. This corrects for QRS complexes that are oriented in the same direction as identified PVCs but are being misidentified as normal QRS complexes due to poor signal quality.

Next, for QRS complex 3 and QRS complex 4, the distance is calculated between each QRS complex's search window and the mean normal QRS complex window. If the difference between this distance and the mean of the normal QRS complex distances is greater than α_{Euclidean} multiplied by the normal QRS complex distance standard deviation, then this QRS complex is changed to a PVC. This corrects for multifocal PVCs.

### QRS Complex Pattern 8—VVNVV Run

According to an embodiment, correlations are calculated between:
QRS complex 3 (N) and QRS complex 1 (V)
QRS complex 3 (N) and QRS complex 2 (V)
QRS complex 3 (N) and QRS complex 4 (V)
QRS complex 3 (N) and QRS complex 5 (V)

If at least three of the correlations between QRS complex 3 and QRS complexes 1, 2, 4, or 5 are greater than 0, QRS complex 3 is changed into a PVC. This corrects for QRS complexes that are oriented in the same direction as identified PVCs but are being misidentified as normal QRS complexes due to poor signal quality.

Following this analysis, the ECG analysis system comprises a final ECG signal classification. The final ECG signal classification may be utilized immediately, and/or it may be stored in local and/or remote memory for future use.

At step 140 of the method, the ECG analysis system provides the final ECG signal classification. According to an embodiment, the ECG analysis system provides the final ECG signal classification to another device or system, such as a patient-monitoring system, an alarm system, or to a database. According to an embodiment, the ECG analysis system provides the final ECG signal classification to a user such as a clinician via a user interface. The system may provide the information to a user via any mechanism, including but not limited to a visual display, a report, a summary, or any other method or mechanism. The information may be communicated by wired and/or wireless communication to another device. For example, the system may communicate the information to a mobile phone, computer, laptop, wearable device, and/or any other device configured to allow display and/or other communication of the information.

At step 150 of the method, a clinician analyzes the provided final ECG signal classification to generate a diagnosis of the patient. This can be performed using a variety of mechanisms, including a manual review of the final ECG signal classification, and/or an automated review of the final ECG signal classification. An experienced clinician can, for example, review the final ECG signal classification and generate a diagnosis for the patient utilizing that experience.

At step 160 of the method, the clinician administers a treatment to the patient based on the diagnosis of that patient (which in turn is based on the final ECG signal classification). The treatment is configured or designed to address the diagnosis, either to correct it or to minimize it, in accordance with an embodiment. There are many treatments available, including but not limited to an arrhythmia treatment medication, cardioversion, ablation, a pacemaker, or other treatment.

Referring again to FIG. 2 is a schematic representation of an ECG analysis system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, system 200 comprises or is in direct or indirect communication with an ECG device 270 configured to obtain ECG data from a subject. The ECG device 270 can be any ECG device, including devices known in the art. The ECG device comprises one or more ECG leads positioned in contact with a subject, according to known methods, and can obtain ECG data from that subject. According to another embodiment, the ECG analysis system 200 comprises or is in direct or indirect communication with a server, database, or other device comprising recorded ECG data.

According to an embodiment, storage 260 of system 200 may store one or more algorithms, modules, and/or instructions to carry out one or more functions or steps of the methods described or otherwise envisioned herein. For example, storage 260 may comprise, among other instructions or data, an ECG signal classification 262, ECG signal analysis instructions 263, and/or reporting instructions 264.

According to an embodiment, each ECG signal classification 262 comprises an ECG signal and a plurality of identified QRS complexes in the ECG signal. The system may obtain an ECG signal using known methods for obtaining ECG signals, which may be a recorded signal. The signal may be obtained continuously or periodically as determined by the settings or parameters of the ECG device or system. Once obtained, the ECG signal may be utilized immediately and/or it may be saved in memory for future analysis. According to an embodiment, the ECG signal classification comprises both the ECG signal and a plurality of QRS complexes identified in the ECG signal. The plurality of QRS complexes may be identified, for example, manually and/or using a QRS complex classification algorithm, among other methods.

According to an embodiment, ECG signal analysis instructions 263 direct the system to analyze an ECG signal classification 262 to identify false positives, false negatives, or other QRS complex misidentifications or misclassifications, as described or otherwise envisioned herein. The analysis results in a final ECG signal classification that can be provided to a clinician for review and patient diagnosis, or can be provided to another device or system for downstream use such as a monitoring system. Thus, the analysis by the ECG analysis system can improve diagnoses and reduce false alarms, among other improvements.

According to an embodiment, reporting instructions 264 direct the system to provide the output of the system to another device or system, or to a user, such as a clinician, via a user interface. The provided output can be any of the information as described or otherwise envisioned herein, including but not limited to the final ECG signal classification. The system may provide the information to a user via any mechanism, including but not limited to a visual display or any other method. The information may be communicated by wired and/or wireless communication to another device. For example, the system may communicate the information to a mobile phone, computer, laptop, wearable device, and/or any other device configured to allow display and/or other communication of the information.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for automated analysis of ECG signal classifications, comprising:
receiving an ECG signal classification for a subject, comprising an ECG signal and a plurality of identified QRS complexes in the ECG signal, wherein at least one of the identified plurality of identified QRS complexes is an erroneous identification or dissimilar from other QRS complexes in the ECG signal;
analyzing the ECG signal classification to identify the at least one erroneous or dissimilar identified QRS complex and generate a final ECG signal classification, comprising: (i) filtering the received ECG signal; (ii) extracting a plurality of features from each of the plurality of identified QRS complexes; (iii) clustering, using the extracted plurality of features, the identified QRS complexes into at least a first cluster of QRS complexes and a cluster of abnormal or dissimilar QRS complexes; (iv) generating a QRS cluster template from the first cluster of QRS complexes; (v) calculating a distance between each of the QRS complexes in the cluster of abnormal or dissimilar QRS complexes and the QRS cluster template; and (vi) classifying, based on the calculated distance, the QRS complexes in the cluster of abnormal or dissimilar QRS complexes as being normal or dissimilar/abnormal to generate the final ECG signal classification; and
reporting the generated final ECG signal classification.

2. The method of claim 1, further comprising the step of identifying, using a QRS complex detection algorithm, a plurality of QRS complexes in an ECG signal.

3. The method of claim 1, wherein filtering the received ECG signal comprises removing baseline wander.

4. The method of claim 1, wherein filtering the received ECG signal comprises bandpass cascaded filtering.

5. The method of claim 1, wherein analyzing the ECG signal classification further comprises reducing dimensionality of extracted features.

6. The method of claim 1, wherein clustering the identified QRS complexes comprises Gaussian mixture modeling and/or hierarchical clustering.

7. The method of claim 1, further comprising:
analyzing the generated final ECG signal classification to generate a diagnosis of the patient; and
administering, based on the diagnosis of the patient, a treatment to the patient configured to address the diagnosis.

8. The method of claim 7, wherein the treatment is one or more of an arrhythmia treatment medication, cardioversion, ablation, a pacemaker, or other treatment.

9. A system for automated analysis of ECG signal classifications, comprising:
an ECG signal classification for a subject, comprising an ECG signal and a plurality of identified QRS complexes in the ECG signal, wherein at least one of the identified plurality of identified QRS complexes is an erroneous identification or dissimilar from other QRS complexes in the ECG signal;
a processor configured to analyze the ECG signal classification to identify the at least one erroneous or dissimilar identified QRS complex and generate a final ECG signal classification, wherein the processor is configured to: (i) filter the received ECG signal; (ii) extract a plurality of features from each of the plurality of identified QRS complexes; (iii) cluster, using the extracted plurality of features, the identified QRS complexes into at least a first cluster of QRS complexes and a cluster of abnormal or dissimilar QRS complexes; (iv) generate a QRS cluster template from the first cluster of QRS complexes; (v) calculate a distance between each of the QRS complexes in the cluster of abnormal or dissimilar QRS complexes and the QRS cluster template; and (vi) classify, based on the calculated distance, the QRS complexes in the cluster of abnormal or dissimilar QRS complexes as being normal or abnormal/dissimilar to generate the final ECG signal classification; and
a user interface configured to provide the generated final ECG signal classification.

10. The system of claim 9, wherein the processor is further configured to identify, using a QRS complex detection algorithm, a plurality of QRS complexes in an ECG signal.

11. The system of claim 9, wherein the filtering the received ECG signal comprises removing baseline wander.

12. The system of claim 9, wherein filtering the received ECG signal comprises bandpass cascaded filtering.

13. The system of claim 9, wherein analyzing the ECG signal classification further comprises reducing dimensionality of extracted features.

14. The system of claim 9, wherein clustering the identified QRS complexes comprises Gaussian mixture modeling and/or hierarchical clustering.

15. The system of claim 14, further comprising:
a diagnosis of the patient based on an analysis of the generated final ECG signal classification; and
a treatment administered to the patient and based on the diagnosis of the patient, wherein the treatment is one or more of an arrhythmia treatment medication, cardioversion, ablation, a pacemaker, or other treatment.
